(19) United States
(12) Patent Application Publication
(10) Pub. No.: US 2019/0174718 A1
(43) Pub. Date: Jun. 13, 2019

(54) DEVICE AND A METHOD OF LEARNING A BEHAVIOR OF A PET IN RESPONSE TO INSTRUCTIONS PROVIDED TO THE PET

(71) Applicant: SMALUET SOLUTIONS PRIVATE LIMITED, Chennai, Tamil Nadu (IN)

(72) Inventors: Ravi Kumar VENKAT, Kochi (IN); Aradhana RAVI, Kochi (IN)

(73) Assignee: SMALUET SOLUTIONS PRIVATE LIMITED, Chennai, Tamil Nadu (IN)

(21) Appl. No.: 16/088,412

(22) PCT Filed: Apr. 6, 2018
(86) PCT No.: PCT/IB2018/052395

§ 371 Date: Sep. 25, 2018

(30) Foreign Application Priority Data
Apr. 7, 2017 (IN) 201741012578

(51) Int. Cl. A01K 15/02; A01K 5/02; A61B 5/11

(52) U.S. Cl. CPC A01K 15/021; A01K 5/0233; A01K 5/0114; A61B 5/1128; A61B 5/1116

(57) ABSTRACT

A device and a method for learning behavior of a pet in response to instructions provided to the pet are disclosed. The device comprises at least one image capturing unit, at least one speaker, and a controller. The at least one speaker announces pre-determined instructions to the pet. The pre-determined instructions are associated with a plurality of pre-defined postures of the pet. The at least one image capturing unit captures postures of the pet in response to announcement of the pre-determined instructions. The controller compares the postures of the pet captured with each of the plurality of pre-defined postures determined to learn behavior of the pet in response to the pre-determined instructions announced.

### FIELD OF INVENTION

The present disclosure relates to a device for learning and training pets. More particularly, the present disclosure relates to a device and a method of learning a behavior of a pet in response to instructions provided to the pet.

### BACKGROUND

Animals play a vital role in many people's lives. Statistical studies have proven that people who raise animals/birds have decreased stress and blood pressure levels. The importance of pets in the society is increasing because of its proven therapeutic benefits for the people such as occupational therapy, speech therapy, etc. As the pets are not capable of understanding human language, pet owners need to provide training and may face difficulty while training. Therefore, pet owners opt for training their pets through professional trainers. This sometimes end up with high cost investment which a common man may not afford. The pets lead a healthy life style if they are trained properly. The pets live happily when they are taught basic discipline and behavior while they live with human beings.

Conventionally, when a trainer sees the pet doing a particular action such as sit, stay, come, down etc., he/she speaks out the word they want the pet to associate its action with and then give it a reward, which can either be its favorite food or a sound generated by a clicker. This could be followed by a pat at its back with a positive word. This process has to be performed multiple times for the pet to obey the command which not only takes long time, but also are tedious and expensive. There are many training systems or devices available to train pets.

For example, US20160270369 discloses a pet feeding training device includes housing, a food delivery mechanism and a food delivery control mechanism. The food delivery control mechanism comprises a token dropping opening, a swinging plate which is able to return to an initial position disposed under the token dropping opening, a food blocking plate provided in the food channel for blocking the food from moving from the food storage compartment to the food outlet, and a driving mechanism, which can lower the food blocking plate and unblock the food channel in response to swinging of the swinging plate. The food in the food channel can be unblocked and delivered from the food outlet only after tokens are thrown into the token dropping opening. This device trains the pet with throwing activity only. However, this does not provide training to pets on responding with the behavioral commands.

Therefore, there exists a need to develop a device to train the pets with basic and complex behavioral commands, wherein a device working based on the camera data and machine learning techniques will enrich the pets, keep them motivated and self-trained.

### SUMMARY

The following summary is illustrative only and is not intended to be in any way limiting. In addition to the illustrative aspects, example embodiments, and features described above, further aspects, example embodiments, and features will become apparent by reference to the drawings and the following detailed description.

One or more of the problems/limitations of the conventional prior art may be overcome by various embodiments of the present invention.

Accordingly, it is the primary object of the present invention to learn a behavior of a pet in response to instructions provided to the pet and then train the pet.

In one aspect of the present disclosure, a method of learning a behavior of a pet in response to instructions provided to the pet is disclosed. The method comprises determining, by a controller, a plurality of pre-defined postures of a pet corresponding to pre-determined instructions. The method further comprises announcing, by at least one speaker, the pre-determined instructions to the pet. The method further comprises capturing, by at least one image capturing unit, postures of the pet in response to announcement of the pre-determined instructions. The method further comprises comparing, by the controller, the postures of the pet captured with each of the plurality of pre-defined postures determined to learn a behavior of the pet in response to the pre-determined instructions announced.

In another aspect of the present disclosure, a device for learning behaviour of a pet in response to instructions provided to the pet is disclosed. The device comprises at least one image capturing unit, at least one speaker, and a controller. The at least one speaker announces pre-determined instructions to the pet. The pre-determined instructions are associated with a plurality of pre-defined postures of the pet. The at least one image capturing unit captures postures of the pet in response to announcement of the pre-determined instructions. The controller compares the postures of the pet captured with each of the plurality of pre-defined postures determined to learn behaviour of the pet in response to the pre-determined instructions announced.

### BRIEF DESCRIPTION OF THE FIGURES

These and other features, aspects, and advantages of the example embodiments will become better understood when the following detailed description is read with reference to the accompanying drawings in which like characters represent like parts throughout the drawings.

FIG. 1A and 1B illustrate an exploded view and a perspective view of a device for learning a behavior of a pet in response to instructions provided to the pet, in accordance with one embodiment of the present disclosure;

FIG. 2 illustrates the device capturing postures of the pet, in accordance with one embodiment of the present disclosure;

FIG. 3A and 3B illustrate a perspective view and an exploded view of a device for learning a behavior of a pet in response to instructions provided to the pet, in accordance with another embodiment of the present disclosure; and

FIG. 4 illustrates a method for learning a behavior of a pet in response to instructions provided to the pet, in accordance with one embodiment of the present disclosure.

Further, skilled artisans will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present invention so as not to obscure the figures with details that will be readily apparent to those of ordinary skill in the art having the benefit of the description herein.

### DETAILED DESCRIPTION

For the purpose of promoting an understanding of the principles of the invention, reference will now be made to the embodiments illustrated in the figures and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

The present disclosure discloses a device and a method of learning a behavior of a pet in response to instructions provided to the pet. The device comprises at least one image capturing unit, at least one speaker, and a controller. The at least one speaker announces pre-determined instructions to the pet. The pre-determined instructions are associated with a plurality of pre-defined postures of the pet. The at least one image capturing unit captures postures of the pet in response to announcement of the pre-determined instructions. The controller compares the postures of the pet captured with each of the plurality of pre-defined postures determined to learn behaviour of the pet in response to the pre-determined instructions announced.

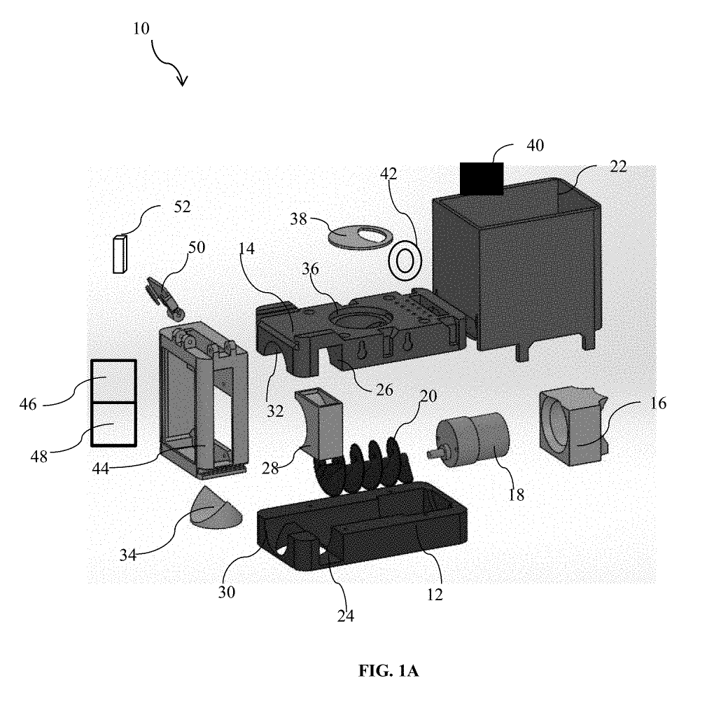

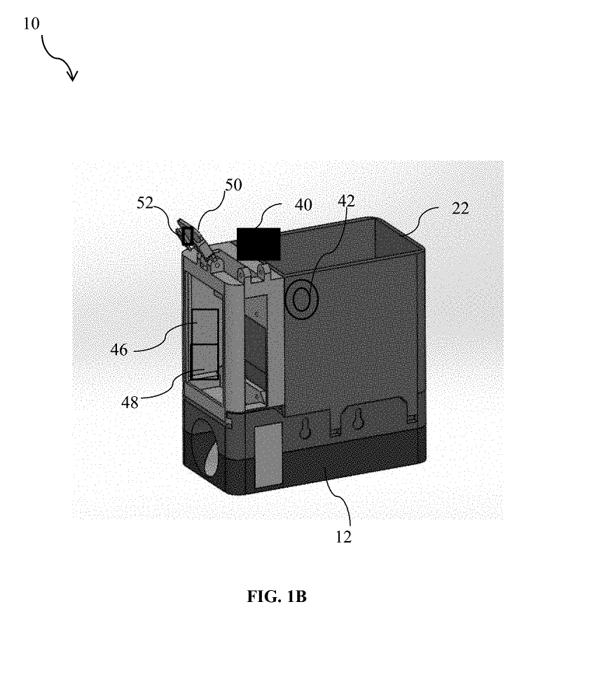

Various embodiments of the device for learning a behavior of a pet are explained using the FIGS. 1A-4.

Now referring to FIG. 1A, an exploded view of a device 10 for learning a behavior of a pet in response to instructions provided to the pet is shown, in accordance with one embodiment of the present disclosure. Further, referring to FIG. 1B, the device 10 when assembled is shown, in accordance with one embodiment of the present disclosure. The device 10 comprises a first housing 12 and a second housing 14. The first housing 12 and the second housing 14 comprises a motor holder 16. The motor holder 16 further comprises a motor 18. The first housing 12 comprises a worm wheel 20 coupled to the motor 18.

Although the present disclosure is presented to include the worm wheel 20, any other suitable mechanical component may be used with a different design which will facilitate in dispensing food to the pet P. The device 10 further comprises a food storage unit 22. The food storage unit 22 is used to store food for a pet. In one implementation, the first housing 12 is provided with a first cut portion 24 and the second housing 14 is provided with a second cur portion 26 to place a first stopper 28. Further, the first housing 12 is provided with a third cut portion 30 and the second housing 14 is provided with a fourth cut portion 32 to place a second stopper 34. Furthermore, the second housing 14 is provided with a fifth cut section 36 to place a food dispensing unit 38. The food dispensing unit 38 is operationally coupled to the food storage unit 22. As such, the food dispensing unit 38 is operated to dispense food from the food storage unit 22.

In one implementation, the food storage unit 22 comprises a speaker 40 provided at outer surface of the device 10. The speaker 40 is used to announce commands or instructions stored in the device 10. Further, the food storage unit 22 comprises an audio receiver 42 provided at outer surface. The audio receiver 42 is used to capture sound made by the pet.

Further, the device 10 comprises a third housing 44. The third housing 44 comprises a controller 46 and a memory 48. The controller 46 may be implemented as one or more microprocessors, microcomputers, microcontrollers, digital signal processors, central processing units, state machines, logic circuitries, and/or any devices that manipulate signals based on operational instructions. Among other capabilities, the controller 46 is configured to fetch and execute computer-readable instructions stored in the memory 48.

The memory 48 may include any computer-readable medium known in the art including, for example, volatile memory, such as static random access memory (SRAM) and dynamic random access memory (DRAM), and/or non-volatile memory, such as read only memory (ROM), erasable programmable ROM, flash memories, hard disks, optical disks, and magnetic tapes.

Furthermore, the device 10 comprises a fourth housing 50. The fourth housing 50 comprises an image capturing unit 52. The image capturing unit 52 captures postures of the pet using known image capturing techniques.

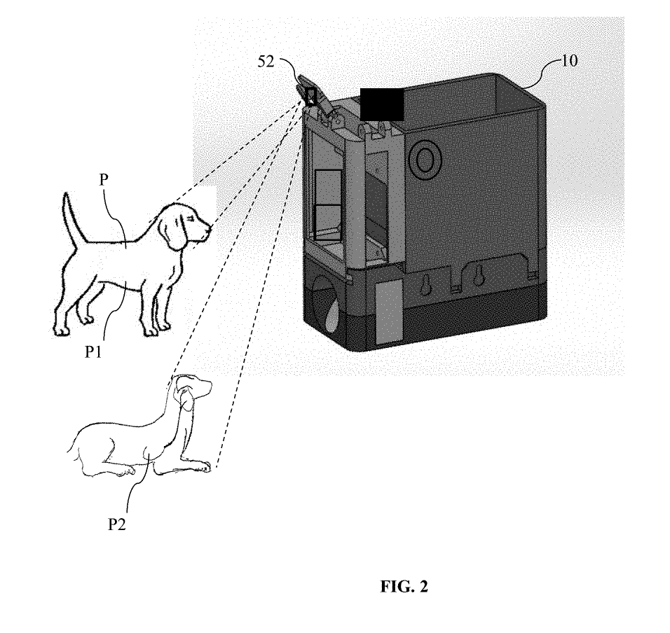

The device 10 is configured to provide instructions to a pet. Specifically, the device 10 is configured to provide the instructions to the pet and to learn a behavior of the pet in response to the instructions provided. Referring to FIG. 2, the device 10 used to learn behavior of a pet P is shown. Examples of pet P may include, but not limited to a cat, dog, and so on. At first, the device 10 is configured to store a plurality of postures of the pet each representing a pre-defined posture of the pet P. The pre-defined postures may include but not limited to sitting posture, standing posture, running posture, or the like. In order to capture the pre-defined postures, the device 10 may employ the image capturing unit 52. The image capturing unit 52 may include a camera that can be used to capture or record postures of the pet P. Referring to FIG. 2, pre-defined posture P1 may indicate a posture for standing. Further, pre-defined posture P2 may indicate a posture for sitting. Similarly, other pre-defined postures are captured. After capturing, each of the pre-defined postures is labeled with pre-determined instructions. In other words, each of the pre-defined postures is tagged with corresponding instructions. For example, the posture P1 is tagged as standing posture. Further, the posture P2 is tagged as sitting posture. Similarly, other pre-defined postures are tagged.

After determining the plurality of pre-defined postures of the pet P corresponding to the pre-determined instructions, the pre-determined instructions are stored in the memory 48.

In one implementation, the device 10 is configured to constantly monitor postures of the pet P. In order to monitor the postures of the pet P, the device 10 may employ the image capturing unit 52. In one example, the device 10 is configured to make an announcement or noise and dispense food using the food dispensing unit 38 upon detecting that the pet P has come to a predefined posture. In order to explain the above aspect, an example may be used. For example, consider that the device 10 is configured to identify sitting posture of the pet P. In order to identify, sitting postures of the pet is stored in the memory 48. The device 10 constantly monitors the pet P until the pet P sits. After identifying that the pet P sat down, the device 10 may be configured to perform additional functions. For example, the device 10 may be configured to dispense food. In another example, the device 10 may be configured to make an announcement.

Considering the above example, the device 10 may be configured to dispense food and make an announcement say a voice clip is played indicating food is being served when the device 10 detects that the pet P changed the posture to sitting posture. The above process is repeated until the pre-determined time the device 10 is configured. Similarly, the device 10 may be configured to recognize postures as may be defined at the beginning and upon detecting change in posture, the device 10 may dispense food, or make an announcement or perform both. Each time the device 10 detects the sitting posture of the pet P, the device 10 captures the position of the pet and the positions are used to classify the sitting postures in subsequent posture identification.

In one implementation, the device 10 is configured to announce the pre-determined instructions to the pet P. Specifically, the speaker 40 is used to announce the pre-determined instructions to the pet P. For example, the speaker 40 is used to announce the pre-determined instruction such as sit to the pet P. In one implementation, the device 10 is configured to announce the pre-determined instruction such as sit to the pet at a specified time e.g., at an interval of 2 hours. At the specified time, the controller 46 may fetch the instructions from the memory 48 and may employ the speaker 40 to announce the pre-determined instruction such as run to the pet P.

After announcing the pre-determined instruction to the pet P, response of the pet P is captured. In one example, the response may be captured using the image capturing unit 52. In another example, the response may be captured using the audio receiver 42. In another example, the response may be captured using the audio receiver 42 and the image capturing unit 52. It is to be understood that the image capturing unit 52 is used to capture the posture of the pet P in response to the announcement of the pre-determined instructions. Further, the audio receiver 42 is used to capture sounds made by the pet P in response to the announcement of the pre-determined instructions.

The response of the pet P to the pre-determined instructions is captured and the response is compared with the pre-determined postures. Specifically, the controller 46 compares the postures of the pet captured with each of the plurality of pre-defined postures determined. For example, consider that the pre-determined instructions e.g., sit is provided at 9 AM to the pet P, the response of the pet P is captured using the image capturing unit 52. Considering that the pet P sits after announcing the pre-determined instructions e.g., sit then the image capturing unit 52 captures the response and sends the response to the controller 46. The controller 46 compares the response (posture) of the pet P with the pre-defined postures i.e., sit, stand and so on. If the posture of the pet P (sit) in response to the announcement of the pre-determined instructions matches with the pre-defined posture (sit) determined corresponding to the pre-determined instructions, then the controller 46 determines that the pet P is following the pre-determined instructions provided to the pet P.

If the posture of the pet P after announcing the pre-determined instructions matches with the pre-defined posture, then the controller 46 initiates the motor 18. After initiating the motor 18, the motor 18 engages the worm wheel 20 to draw food stored in the food storage unit 22 and dispenses through the food dispense adjuster 38. In one example, the controller 46 may control the amount of food to be delivered to the pet P when the posture of the pet P matches with the pre-defined postures corresponding to pre-determined instructions provided to the pet P. In order to control the amount the food to be delivered to the pet P, the controller 46 may utilize the first stopper 28, the second stopper 34 and the food dispensing adjuster 28. In one example, the amount of food to be delivered at the given time is configured in the device 10. Further, the device 10 is configured to send alerts to a pet owner when the food in the food storage unit 22 falls below certain level.

In another example, consider that the device 10 announces a pre-determined instruction e.g., sit at 10 AM. After announcing the pre-determined instruction, consider that the pet P does not sit and remains in other posture e.g., stand or running. In the above example, the device 10 employs the image capturing unit 52 to capture posture of the pet P i.e., standing in response to the pre-determined instruction of sit. Subsequently, the posture of the pet P i.e., stand is compared with the pre-defined postures of pet P corresponding to pre-determined instruction of sit. If the posture of the pet P after announcement does not match with the pre-defined postures corresponding to pre-determined instructions, then the controller 46 records the behavior of the pet P. If the posture of the pet P does not match, then the controller 46 may not dispense the food from the food storage unit 22.

In one example, the device 10 may be configured to play voice recordings or music in response to the postures of the pet P matching with the pre-defined postures corresponding to pre-determined instruction announced. The voice recordings may be played in appreciation of the pet P following the pre-determined instructions announced. Further, the response of the pet P for the appreciation provided is captured to learn the behavior of the pet P.

As explained above, the device 10 is configured to announce the pre-determined instructions to the pet P at pre-defined intervals, capture the posture of the pet P and compare the posture of the P with the pre-defined postures corresponding to the pre-determined instructions. For each of the announcement, the device 10 captures the response of the pet P and stores the response in the memory 48. Based on the response of the pet P, a behavior pattern of the pet P may be captured. For instance, the response the pet P corresponding to the pre-determined instructions provided in a day may be captured to determine the behavior pattern of the pet P. In another example, the response the pet P corresponding to the pre-determined instructions provided in a week or a month may be captured to determine the behavior pattern of the pet P.

In one implementation, the behavior pattern captured may be used to reconfigure the pre-determined instructions provided to the pet P. For example, consider that the pet P is not responding to the pre-determined instruction such as sit at a specific time of the day say at 12 PM. For the above example, the device 10 may be reconfigured to announce pre-determined instruction such as stand at 12 PM and the response of the pet P may be compared with the pre-determined postures corresponding to the pre-determined instruction reconfigured.

Based on the response of the pet P, the behavior pattern of the pet P is learned. In one implementation, the device 10 may be used to train the pet P based on the behavior pattern learned. For example, if the pet P is determined to follow certain type of pre-determined postures such as sit and stand and not running, then the device 10 may be reconfigured to announce the pre-determined instructions such as run more often such that the pet P may be trained to run in response to the pre-determined instruction i.e., run.

Furthermore, the device 10 may be configured to provide various other instructions associated with different postures such that the pet P may be trained to behave in certain manner.

In one implementation, the device 10 may comprise a visible indicator (not shown) to provide visible indications to the pet P. The visible indicator may indicate a laser, or a flash light that may be mounted on a Pan Tilt arrangement at the outer surface of the device 10. The device 10 may be configured to flash the visible indications in front of the pet P such that the pet P may follow the visible indications. The pet P following the visible indications may be captured using the image capturing unit 52. If the pet P is following the visible indications, then the device 10 may dispense the food from the food dispensing unit 22. Based on the response of the pet P, the behavior of the pet P following the visible indications provided may be learnt and the visible indications may be reconfigured to train the pet P.

The device 10 is used to train the pet with basic and/or complex behavioral commands to improve behavior of the pet. Further, the behavior of the pet is captured over a period of time to reconfigure the instructions provided to the pet such that the pet may be trained. Based on the response of the pet, food may be dispensed from the food storage unit provided in the device or the pet may be provided with rewards in the form of voice recordings, playing music and so on. The device may be reconfigured to learn the behavior of the pet and train the pet.

In another implementation, the device 10 may be used to capture various positions of the pet P in sitting posture when the announcement was made and store in the memory 48.

The various positions of the pet P in sitting posture is captured to compare position of the pet P when announcements are made at subsequent instances. As such, the device 10 compares the posture of the pet P with variety of positions and improves the accuracy of the matching the sitting posture with pre-stored postures corresponding to the announcement. The above procedure is repeated using a machine learning algorithm to improve the accuracy of the device 10 recognizing, classifying and matching the posture of the pet P after making the announcement with pre-stored postures.

In one implementation, the device 10 may be used to capture behavior of the pet P when a person rings a door bell of the house indicating that a person living in the house has come home. After the bell rings, the behavior of the pet P may be captured. For example, if the pet P is sitting at one place without jumping on to the person who entered house, then the device 10 may dispense a reward for the pet P. Further, if the device 10 detects that the pet P jumped on the person, then a warning alert may be announced by the device 10 indicating that the pet P not to jump on the person. The device 10 leans the behavior of the pet P over a period of time and configured further instructions that needs to be given to the pet P.

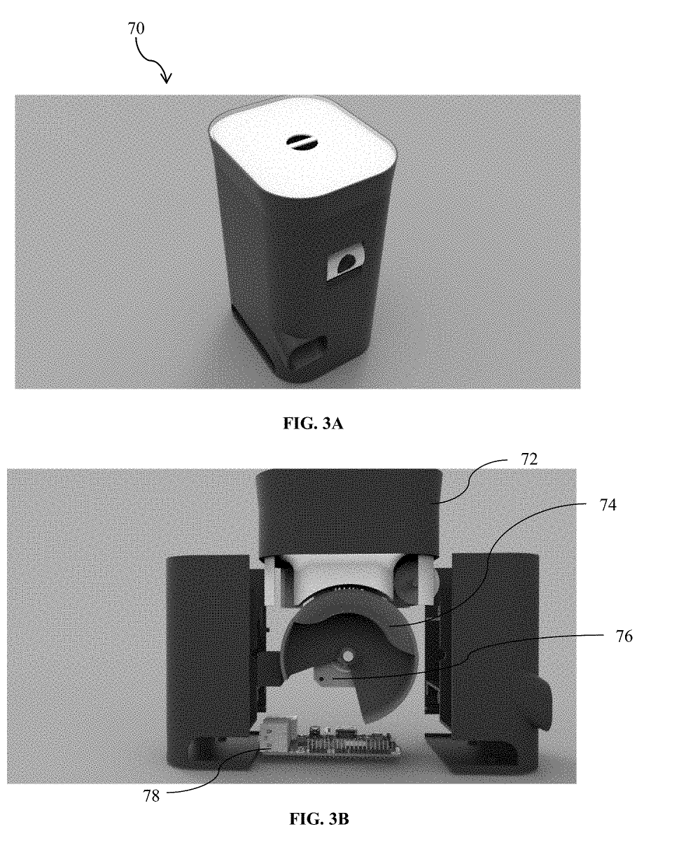

Referring to FIG. 3, a device 70 used for learning a behavior of a pet in response to instructions provided to the pet is shown, in accordance with an alternate embodiment of the present disclosure. The device 70 comprises a food storage unit 72 used to store food for the pet. The device 70 further comprises a food dispenser unit 74 (similar to food dispensing unit 38 in FIG. 1) operationally coupled to the food storage unit 72. The device 70 further comprises a motor 76 to operate the food dispenser unit 76. Furthermore, the device 70 comprises a controller 78 (similar to controller 46 in FIG. 1). The device 70 may comprise an image capturing unit (not shown) to capture or record postures of the pet. Based on the postures captured by the image capturing unit, the controller 78 controls operation of the motor 76. As such, the motor 76 is operated to open the food storage unit 72 in order to dispense the food into the food dispenser unit 76 which in turn dispenses the food outside of the device 70 for the pet.

FIG. 3 is shown to present an alternate design of the device in which the device used for learning a behavior of a pet can be used without use of worm wheel as shown in FIG. 1.

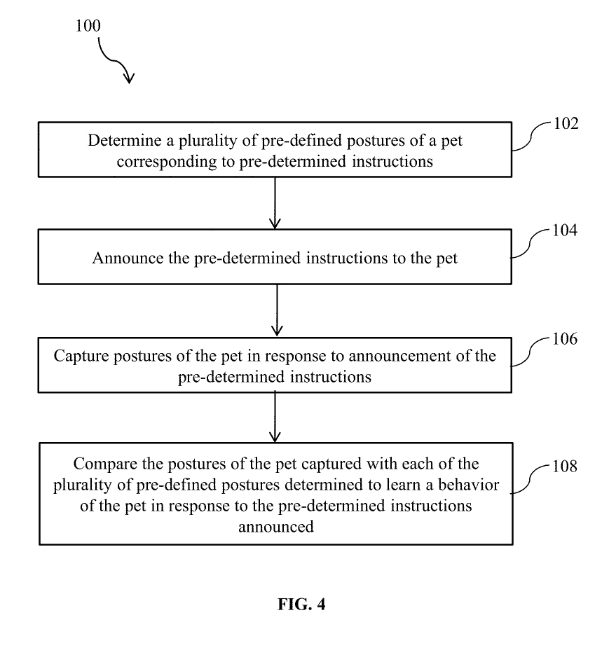

Referring now to FIG. 4, a method 100 for learning a behaviour of a pet in response to instructions provided to the pet is shown, in accordance with an embodiment of the present disclosure. The method 100 may be described in the general context of computer executable instructions. Generally, computer executable instructions may include routines, programs, objects, components, data structures, procedures, modules, functions, etc., that perform particular functions or implement particular abstract data types.

The order in which the method 100 is described and is not intended to be construed as a limitation, and any number of the described method blocks can be combined in any order to implement the method 100 or alternate methods. Additionally, individual blocks may be deleted from the method 100 without departing from the spirit and scope of the disclosure described herein. The method 100 is explained with reference to the device 10 explained above.

At step 102, a plurality of pre-defined postures of a pet corresponding to pre-determined instructions is determined. In one example, the plurality of pre-defined postures is determined using the controller 46.

At step 104, the pre-determined instructions to the pet are announced. In one example, the pre-determined instructions are announced using the speaker 40.

At step 106, postures of the pet in response to announcement of the pre-determined instructions are captured. In one example, the postures of the pet are captured using the image capturing unit 42.

At step 108, the postures of the pet captured are compared with each of the plurality of pre-defined postures determined to learn a behaviour of the pet in response to the pre-determined instructions announced. In one example, the postures of the pet captured are compared using the controller 46.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible.

### REFERENCE NUMERALS

Device 10

First Housing 12

Second Housing 14

Motor Holder 16

Motor 18

Worm Wheel 20

Food Storage Unit 22

First Cut Portion 24

Second Cut Portion 26

First Stopper 28

Third Cut Portion 30

Fourth Cut Portion 32

Second Stopper 34

Fifth Cut Section 36

Food Dispensing Unit 38

Speaker 40

Audio Receiver 42

Third Housing 44

Controller 46

Memory 48

Fourth Housing 50

Image Capturing Unit 52

Device 70

Food Storage Unit 72

Food Dispenser Unit 74

Controller 78

We claim:

1. A method (100) of learning a behaviour of a pet in response to instructions provided to the pet, the method comprising:
determining (102), by a controller, a plurality of pre-defined postures of a pet corresponding to pre-determined instructions;
announcing (104), by at least one speaker, the pre-determined instructions to the pet;
capturing (106), by at least one image capturing unit, postures of the pet in response to announcement of the pre-determined instructions; and
comparing (108), by the controller, the postures of the pet captured with each of the plurality of pre-defined postures determined to learn a behaviour of the pet in response to the pre-determined instructions announced.

2. The method (100) as claimed in claim 1, further comprises:
dispensing food when the posture of the pet matches with one of the plurality of pre-defined postures determined in response to pre-determined instructions.

3. The method (100) as claimed in claim 1, further comprises:
capturing, by an audio receiver, sound made by the pet in response to announcement of the pre-determined instructions.

4. The method (100) as claimed in claim 1, further comprises:
determining, by the controller, a behaviour pattern of the pet based on the postures captured in response to announcement of the pre-determined instructions.

5. The method (100) as claimed in claim 4, further comprises:
reconfiguring, by the controller, the pre-determined instructions based on the behaviour pattern determined to train the pet.

6. A device (10) for learning a behaviour of a pet P in response to instructions provided to the pet P, the device (10) comprising:
at least one image capturing unit (42);
at least one speaker (40); and
a controller (42);
wherein the at least one speaker (40) announces pre-determined instructions to the pet P, wherein the pre-determined instructions are associated with a plurality of pre-defined postures of the pet P,
wherein the at least one image capturing unit (42) captures postures of the pet P in response to announcement of the pre-determined instructions, and
wherein the controller (46) compares the postures of the pet P captured with each of the plurality of pre-defined postures determined to learn a behaviour of the pet P in response to the pre-determined instructions announced.

7. The device (10) as claimed in claim 6, further comprises an audio receiver (42) to capture sound made by the pet in response to announcement of the pre-determined instructions.

8. The device (10) as claimed in claim 6, further comprises a food storage unit (22) to store food for the pet.

9. The device (10) as claimed in claim 8, further comprises a food dispensing adjuster (38) coupled to the food storage unit (22), wherein the controller (46) controls dispensing of the food based on the posture of the pet in response to announcement of the pre-determined instructions.

10. The device (10) as claimed in claim 6, wherein the controller (46) determines a behaviour pattern of the pet based on the postures captured in response to announcement of the pre-determined instructions, and wherein the controller (46) reconfigures the pre-determined instructions based on the behaviour pattern determined to train the pet.

11. The device (10) as claimed in claim 6, further comprises a visible indicator to provide visible indications to the pet.

12. The device (10) as claimed in claim 11, wherein the controller (46) learns the behaviour of the pet following the visible indications provided to the pet.
